SEQUENCE LISTING (1 sequence)

SEQ ID NO: 1 - DNA, 556 bp, unidentified
  source 1..556: mol_type = other DNA; note = Aureobasidium pullulans
taggagataagggtgctcagcgcccgacctccaaccctttgttgttaaaactaccttgttgctttggcgggaccgctcggtctcgagccgctggggattcgtcccaggcgagcgcccgccagagttaaaccaaactcttgttatttaaccggtcgtctgagtaaaaattttgaataaatcaaaactttcaacaacggatctcttggttctcgcatcgatgaagaacgcagcgaaatgcgataagtaatgtgaattgcagaattcagtgaatcatcgaatctttgaacgcacattgcgccccttggtattccgaggggcatgcctgttcgagcgtcattacaccactcaagctatgcttggtattgggcgtcgtccttagttgggcgcgccttaaagacctcggcgaggccactccggctttaggcgtagtagaatttattcgaacgtctgtcaaaggagaggaactctgccgactgaaacctttatttttctaggttgacctcggatcaggtagggatacccgctgaacttaagcatatcaaaagccgggaggaaa

(19) United States
(12) Patent Application Publication
(10) Pub. No.: US 2025/0002966 A1
(43) Pub. Date: Jan. 2, 2025

(54) AUREOBASIDIUM PULLULANS P1 AND METHOD FOR PREPARING RARE GINSENOSIDE

(71) Applicant: Shaanxi Institute of Microbiology, Xi’an (CN)

(72) Inventors: Wenjiao Xue, Xi’an (CN); Chao An, Xi’an (CN); Saijian Ma, Xi’an (CN); Qiwen Zhang, Xi’an (CN); Hao Ding, Xi’an (CN); Chen Liu, Xi’an (CN); Yao Liu, Xi’an (CN); Jingjing Zhang, Xi’an (CN)

(21) Appl. No.: 18/772,177

(22) Filed: Jul. 14, 2024

(51) Int. Cl. C12P 33/20; C12N 1/14; C12N 9/34; C12R 1/645

(52) U.S. Cl. CPC C12P 33/20; C12N 1/145; C12N 9/2428; C12Y 302/01003; C12R 2001/645

(57) ABSTRACT

An aureobasidium pullulans P1 and application thereof in rare saponin conversion, and belongs to the technical field of microorganisms and biological medicines. The strain preservation number is CGMCC No.23227. The Aureobasidium pullulans P1 can be used for preparing rare ginsenoside Rg3 and CK by converting ginsenoside Rb1 by beta-glycosidase through fermentation, the target products are various and definite, other impurities have small interference, the products are easy to generate, the conversion specificity is high, the efficiency is high, the product recovery rate is over 60%, and the method has obvious advantages in the process of producing Rg3 and CK by mass fermentation.

### SEQUENCE LISTING

The substitute sequence listing is submitted as a XML file filed via EFS-Web, with a file name of “Substitute_Sequence_Listing.XML”, a creation date of Sep. 14, 2024, and a size of 2,648 bytes. The substitute sequence Listing filed via EFS-Web is a part of the specification and is incorporated in its entirety by reference herein.

### TECHNICAL FIELD

The invention relates to the technical field of microorganisms and biological medicines, in particular to an Aureobasidium pullulans P1 and application thereof in preparing rare ginsenoside by converting ginsenoside Rb1.

### BACKGROUND ART

Ginsenoside is triterpene glycoside compound, and is a main medicinal active ingredient of Ginseng radix, and is classified into main ginsenoside (such as Rb1, Rb2, Rc, Rd, Re, Rg1, etc. accounting for more than 80% of total saponins) and Rare ginsenoside (such as Rg3, Rh1, Rh2, F1, F2, CK, C-O, CY, C-Mc), etc. according to its content in plant. The rare ginsenoside refers to the saponin with extremely low or no content in the ginseng, but the pharmacological activity of the rare ginsenoside is superior to that of the high-content ginsenoside, for example, the rare ginsenoside Rg3 has various pharmacological activities of resisting tumor, preventing and treating cardiovascular and cerebrovascular diseases and coronary heart disease, inhibiting cancer cell metastasis, protecting liver, protecting nerves, improving immunity and the like. The rare ginsenoside CK has good anti-cell mutation, tumor cell metastasis inhibiting, tumor cell apoptosis inducing, tumor cell drug resistance reversing, antiallergic and anti-inflammatory activities.

Although ginsenoside Rg3 and CK have high medicinal value, they are very rare in nature, and are difficult to separate from ginseng and other plants by a traditional method, which causes great limitation on the production of rare ginsenosides. Currently, the methods for preparing rare ginsenoside are mainly a direct acid hydrolysis method and a microorganism/enzyme conversion method. Direct acid hydrolysis is generally poor in selectivity, low in yield, not easy to purify, and easy to cause environmental pollution. The microorganism/enzyme conversion method has the advantages of high regioselectivity, stereoselectivity, high group selectivity, good targeting property, high yield, few byproducts, no pollution, easy industrial production and the like, can complete some reactions which are difficult to carry out by chemical methods, and obtains rare ginsenoside such as Rg3 and CK which are difficult to obtain by chemical methods, thus being considered as the most potential method for producing the rare ginsenoside.

The ginsenoside is used as a substrate to be converted into specific rare ginsenoside, so that the resource utilization rate can be improved, the extraction cost of the ginsenoside raw material can be reduced, but the specificity of various enzymes to the substrate is different, and the types of degraded glycosidic bonds are different. The strain capable of converting ginsenoside Rb1 and simultaneously generating Rg3 and CK is few, the recovery rate of the product is low, and the practical application of large-scale production of Rg3 and CK cannot be satisfied. Therefore, the search for strains capable of producing glycoside hydrolase with high efficiency and specificity for degrading ginsenoside Rb1 to produce ginsenoside Rg3 and CK by fermentation becomes a key point of research.

### SUMMARY

In view of this, the invention provides an Aureobasidium pullulans P1 and application thereof.

In order to achieve the above purpose, the present invention adopts the following technical scheme.

The Aureobasidium pullulan P1 is preserved in China general microbiological culture Collection center (CGMCC) with the preservation number of CGMCC No.23227 on 2021 Sep. 10, and is classified and named Aureobasidium pullulans by the national institute of microbiology, national institute of sciences, Beichen West road No.1, the Korean region of Beijing.

Another object of the present invention is to provide an application of the aureobasidium pullulans P1 in preparing rare ginsenoside by converting ginsenoside Rb1.

Preferably, the rare ginsenoside is rare ginsenoside Rg3 and rare ginsenoside CK.

Preferably, the conversion of ginsenoside Rb1 to rare ginsenoside is achieved by fermenting Aureobasidium pullulans P1 to produce beta-glucosidase.

Preferably, the conversion path of the ginsenoside Rb1 into rare ginsenoside is Rb1-Rd-Rg3. The conversion route of ginsenoside Rb1 into rare ginsenoside CK is Rb 1-Rd-F2-CK.

Compared with the prior art, the invention discloses and provides the aureobasidium pullulans P strain which can convert main ginsenoside Rb1 into commercial rare ginsenoside Rg3 and CK with high added value, and has the advantages of various and clear target products, small interference of other impurities, easy generation of the products and obvious advantages in the process of producing Rg3 and CK by mass fermentation. In addition, the specificity of transformation is strong, the efficiency of transforming Rb1 into rare ginsenoside is high, and the product recovery rate is more than 60%.

### DETAILED DESCRIPTION OF THE EMBODIMENTS

In order to more clearly illustrate the embodiments of the present invention or the technical solutions in the prior art, the drawings that are required to be used in the embodiments or the description of the prior art will be briefly described below, and it is obvious that the drawings in the following description are only embodiments of the present invention, and that other drawings can be obtained according to the provided drawings without inventive effort for a person skilled in the art.

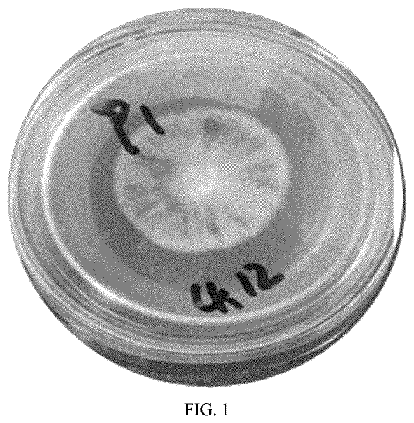

FIG. 1 shows colony morphology characteristics of Aureobasidium pullulans P1 strain;

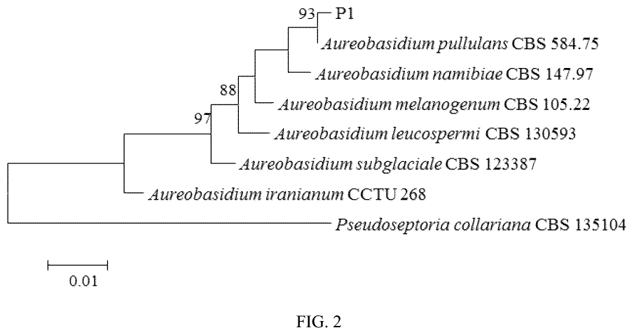

FIG. 2 is a phylogenetic tree construction of Aureobasidium pullulans strain;

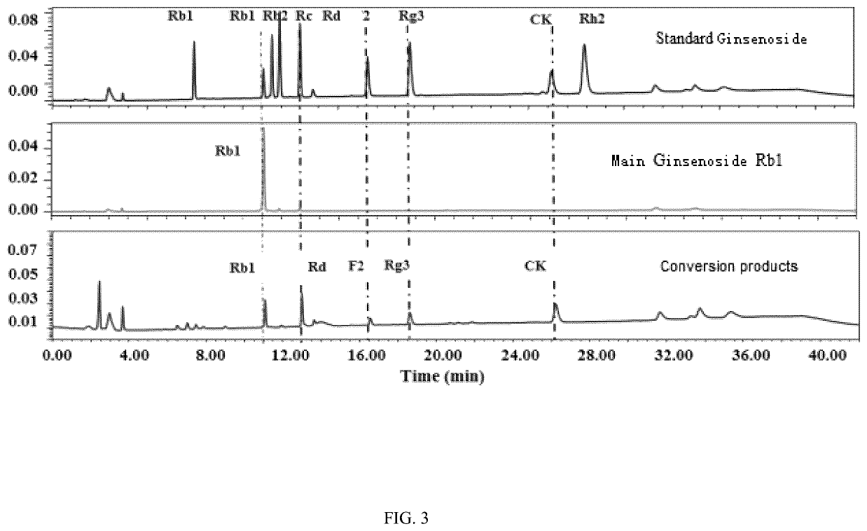

FIG. 3 is an HPLC detection chart of ginsenoside Rb1 and conversion products in fermentation broth before and after fermentation conversion of aureobasidium pullulans P strain;

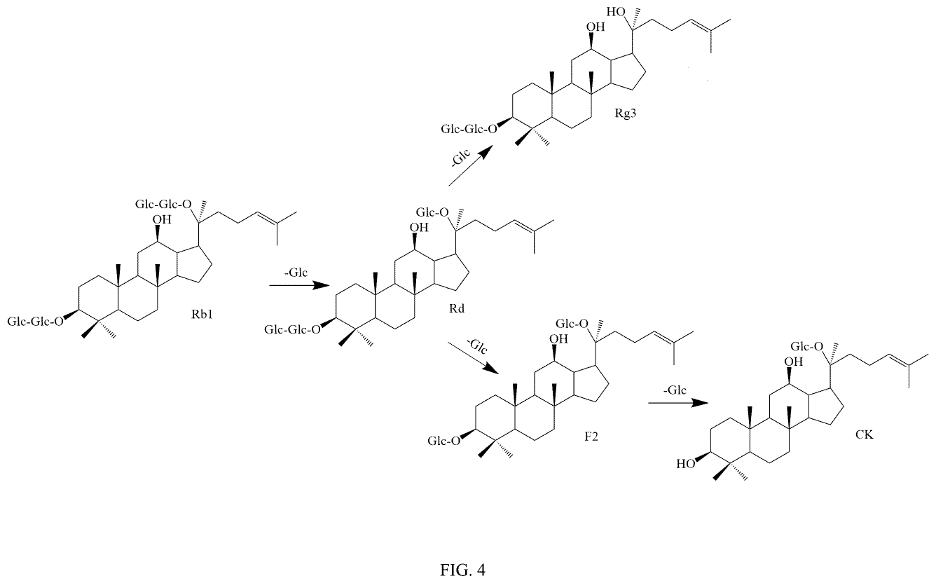

FIG. 4 is a conversion pathway of the main ginsenoside Rb1 to rare ginsenosides;

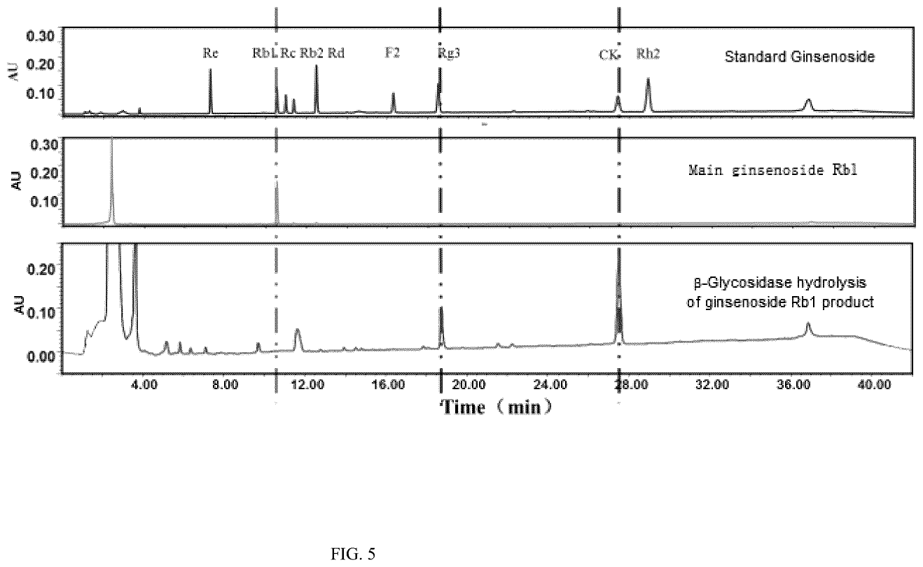

FIG. 5 is an HPLC detection chart of ginsenoside before and after preparing beta-glycosidase by fermenting aureobasidium pullulans P1 and using the glycosidase to convert main ginsenoside Rb1.

### DETAILED DESCRIPTION OF THE EMBODIMENTS

The following description of the embodiments of the present invention will be made clearly and completely, and it is apparent that the described embodiments are only some embodiments of the present invention, but not all embodiments. All other embodiments, which can be made by those skilled in the art based on the embodiments of the invention without making any inventive effort, are intended to be within the scope of the invention.

In order to further understand the present invention, the following details are set forth in the examples, wherein the reagents involved in the examples may be purchased commercially, unless otherwise specified, and the methods not mentioned are conventional experimental methods, and are not described in detail herein.

### EXAMPLE 1

### Identification of Aureobasidium pullulans P1

### 1. Isolation and Culture of Strains

The strain is derived from plant seven endophytic fungi, and the specific culture method includes the steps of cutting root tissues of wild seven of the Qinling mountain into 1 cm, sequentially disinfecting 1 min with 75% ethanol, 2 min with 2.5% sodium hypochlorite, and 1 min with 75% ethanol, washing 3-5 times with sterile water, placing into a culture dish which is sterilized in advance, inserting tissue bloCKs into a PDA culture medium flat plate containing 100 U/mL of acarmiacin sulfate with sterile forceps, and then inversely culturing for 5-7 days in a constant temperature incubator at 28° C. until mycelia grow out.

### 2. Screening and Identification of Strains

Inoculating mycelium grown on a PDA plate to an R2A culture medium containing esculin, and culturing in an inverted manner for 5-7 days in a constant temperature incubator at 28° C., wherein the esculin-R2A agar culture medium includes the following components: 1 g/L esculin, 0.5 g/L ferric citrate, 15.2 g/L R2A commercial medium. The culture medium was sterilized at 121° C . for 20 min.

If the strain is able to produce a beta-glucosidase to hydrolyze esculin, a red halo will be produced around the growth of the strain. After the detection, a strain capable of producing beta-glycosidase is obtained by screening, and is named as a P1 strain, and colony characteristics are shown in FIG. 1.

Sequencing the strain by the HUADA Biotechnology Co., Ltd. The ITS sequence of the strain is shown as SEQ ID NO. 1. Sequencing results Blast similarity analysis was performed in NCBI nucleic acid database, and Blast sequence alignment and treeing analysis (see FIG. 2) were performed to determine that they were Aureobasidium pullulans.

### EXAMPLE 2

### Identification of Fermentation Performance of Aureobasidium pullulans P1 on Rb1

Culturing aureobasidium pullulans P1 strain in PDA culture medium at 28° C. for 5 d, and inoculating the bacterial bloCK into liquid culture solution including the following components: 20 g/L glucose, 10 g/L yeast powder, 0.5 g/L NaNO3, 1 g/L KH2 PO4, 2 g/L Na2HPO4, 0.2 g/L FeSO4, 0.1 g/L ZnSO4, 0.2 g/L CuSO4, 1 g/L CaCl2, 0.5 g/L MgSO4·7H2O. The initial pH was 6.5, and the culture was carried out at a temperature of 28° C. and a rotation speed of 180 rpm for 72 hours.

Dissolving ginsenoside Rb1 with 2% methanol aqueous solution to obtain ginsenoside Rb1 mother solution with concentration of 200 mg/L, filtering, sterilizing, adding into the fermentation broth of aureobasidium pullulans P, fermenting for 72 hr, and culturing for 6 hr; 2 mL of supernatant is taken and added with n-butanol with the volume of 2 times for extraction, then the n-butanol phase at the upper layer is removed to a new culture dish, and the culture dish is placed in a fume hood for volatilizing until the weight is constant, so as to obtain a dry substance.

The dried material was dissolved in methanol and then subjected to liquid phase HPLC detection under the following chromatographic conditions: the chromatographic column adopts YMC-PaCK ODS-AQ with the specification of 250 mm multiplied by 4.6 mm and 5 μm; the mobile phase adopts A phase-B phase, the A phase is water, the B phase is 23 wt % acetonitrile, and the gradient elution mode is as follows: 0 to 12 min, 77 to 48 weight percent of A is adopted; for 12-35 min, 48-25 wt % of A is adopted; 35-36 min, 25 wt % of A is adopted; 36-42 min, 25-77 wt % of A is adopted. The volume flow rate in the gradient elution process is 1 mL/min, the column temperature is 30° C., and the sample injection amount is 10 μL. All samples were subjected to absorbance detection at 203 nm.

HPLC detection is carried out on the strain fermentation Rb1 product, and the strain fermentation Rb1 product is compared with the HPLC spectrogram and data of the standard strain ginsenoside (see FIG. 3). In the conversion product, the content of Rb1 is reduced, the ginsenoside Rd, F2, Rg3 and CK are generated, and the strain can convert the ginsenoside Rb1 into rare saponins Rg3 and CK, and the conversion paths (see FIG. 4) are Rb1, Rd, Rg3 and Rb1, Rd, F2 and CK respectively.

### EXAMPLE 3

### Identification of Rb1 Fermentation Performance of Beta-glycosidase Prepared by Fermentation of Aureobasidium pullulans P1

Aureobasidium pullulans P1 strain is cultivated in PDA culture medium, cultivated for 5 d at 28° C., then the bacterial bloCK is inoculated into liquid culture liquid, and the components of the liquid culture liquid are as follows: 20 g/L glucose, 10 g/L yeast powder, 0.5 g/L NaNO3, 1 g/L KH2PO4, 2 g/L Na2HPO4, 0.2 g/L FeSO4, 0.1 g/L ZnSO4, 0.2 g/L CuSO4, 1 g/L CaCl2, 0.5 g/L MgSO4·7H2O. The initial pH was 6.5, and the culture was carried out at a temperature of 28° C. and a rotation speed of 180 rpm for 96 hours.

Centrifuging the fermentation liquor at 8000 rpm/mm after the culture is finished to remove thalli, adding 70% ammonium sulfate into supernatant to carry out salting out, standing overnight at 4° C. and centrifuging to obtain crude protein, dissolving the crude protein in water, desalting by a dialysis bag, and freeze-drying the desalted enzyme solution to obtain an enzyme sample; respectively dissolving enzyme sample and ginsenoside Rb1with phosphate buffer with pH of 5.0 to obtain 200 mg/L enzyme solution and 200 mg/L ginsenoside Rb1 solution, mixing the enzyme solution and ginsenoside Rb1 solution uniformly in equal volume, and reacting in a water bath at 55° C. for 1 hr. After the reaction, the reaction solution is extracted by 2 times of n-butanol, the n-butanol phase at the upper layer is removed to a new culture dish, and then the culture dish is placed in a fume hood for volatilizing until the weight is constant, so as to obtain a dry product.

The dried material was dissolved in methanol and then subjected to liquid phase HPLC detection under the following chromatographic conditions: the chromatographic column adopts YMC-PaCK ODS-AQ with the specification of 250 mm multiplied by 4.6 mm and 5 μm; the mobile phase adopts A phase-B phase, the A phase is water, the B phase is 23 wt % acetonitrile, and the gradient elution mode is as follows: 0 to 12 min, 77 to 48 weight percent of A is adopted; for 12-35 min, 48-25 wt % of A is adopted; 35-36 min, 25 wt % of A is adopted; 36-42 min, 25-77 wt % of A is adopted. The volume flow rate in the gradient elution process is 1 mL/min, the column temperature is 30° C., and the sample injection amount is 10 mu L. All samples were subjected to absorbance detection at 203 nm.

The HPLC detection is carried out on the strain fermentation Rb1 product, and the strain fermentation Rb1 product is compared with the HPLC spectrogram and data of the ginsenoside in the standard strain (see FIG. 5), in the conversion product, Rb1 is completely degraded, only rare ginsenoside Rg3 and CK are detected, and the recovery rate of the rare ginsenoside Rg3 and CK is obtained by peak area calculation and exceeds 60%.

In the present specification, each embodiment is described in a progressive manner, and each embodiment is mainly described in a different point from other embodiments, and identical and similar parts between the embodiments are all enough to refer to each other. For the device disclosed in the embodiment, since it corresponds to the method disclosed in the embodiment, the description is relatively simple, and the relevant points refer to the description of the method section.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. An aureobasidium pullulans P1 with a preservation number of CGMCC 23227.

2. A method for preparing a rare ginsenoside, comprising:
culturing the aureobasidium pullulans P1 strain of claim 1 in a PDA culture medium;
inoculating the bacterial bloCK into liquid culture solution;
centrifuging the fermentation broth to remove bacterial cells after the cultivation is completed;
adding ammonium sulfate to the supernatant for salt precipitation, and crude protein is obtained by centrifugation; dissolving crude protein in water and desalinating in a dialysis bag;
freeze drying the desalinated enzyme solution to obtain an enzyme sample;
preparing the enzyme sample into an enzyme solution and mixing it with a solution containing ginsenoside Rb1 to obtain the rare ginsenoside.

3. The method of claim 2, wherein the rare ginsenosides are rare ginsenosides Rg3 and rare ginsenosides CK.

4. The method of claim 2, wherein a transformation is effected by fermentation of aureobasidium pullulans P1 to produce β-glucosidase.

5. The method of claim 2, wherein a conversion pathway of ginsenoside Rb1 to rare ginsenoside is Rb1→Rd→Rg3.

6. The method of claim 2, wherein the conversion pathway of ginsenoside Rb1 to rare ginsenoside CK is Rb1→Rd→F2→CK.
